(19) United States
(12) Patent Application Publication
(10) Pub. No.: US 2011/0294155 A1
(43) Pub. Date: Dec. 1, 2011

(54) MODULATION OF AN ION CHANNEL OR RECEPTOR

(75) Inventors: Steven Petrou, Carlton (AU); Even Alenxander Thomas, Carlton (AU)

(73) Assignee: Howard Florey Institute, Carlton (AU)

(21) Appl. No.: 13/131,452

(22) PCT Filed: Nov. 27, 2009
(86) PCT No.: PCT/AU2009/001552

§ 371 Date: Aug. 17, 2011

(30) Foreign Application Priority Data
Nov. 27, 2008 (AU) 2008906148

(51) Int. Cl. C12Q 1/02; G06F 19/10; C12M 1/34

(52) U.S. Cl. 435/29; 435/287.1; 702/19

(57) ABSTRACT

This invention relates to a method of assaying a compound for its ability to modulate an ion channel or receptor type, the method comprising: a) providing a dynamic clamp in electrical contact with a biological cell (or part thereof) in which one or more ion channel or receptor types for providing a waveform are functional and in which one or more ion channel or receptor types for providing a waveform are either not present or not functional; b) causing the dynamic clamp to apply a signal simulating the function of at least one of the one or more ion channel or receptor types that are either not present or not functional in the biological cell (or part thereof) based on modulation of the ion channel or receptor types that are functional in the biological cell (or part thereof) to thereby provide the waveform at the biological cell (or part thereof); c) exposing at least one of the one or more functional ion channel or receptor types to a compound; and d) detecting modulation of the waveform at the biological cell (or part thereof), wherein modulation of the waveform is indicative of a compound that modulates the at least one functional ion channel or receptor types.

### FIELD OF THE INVENTION

The present invention relates to methods of assaying compounds that modulate one or more ion channels or receptors that are involved in providing a waveform at a biological cell, and also to apparatuses and processes for performing such assays. The present invention especially relates to the use of a dynamic clamp in such assays.

### BACKGROUND OF THE INVENTION

In many living organisms signals are transmitted between cells, such as neurons and muscle cells, by variations across cell membranes in electrophysiological parameters such as voltage, current or capacitance. Variations in such electrophysiological parameters often involve large numbers of multiple types of ion channels or receptors, which together produce a waveform at the biological cell. An action potential is an example of one type of waveform.

The waveform results from modulation of ion channels or receptors at the cell. For example, these ion channels or receptors may regulate the transmembrane and intercellular movement of physiological ions, such as Na⁺, K⁺, Ca²⁺, and Cl⁻, which form part of the signal. Modulation of one, or a group of ion channels or receptors results in electrophysiological changes at the membrane of the cell, causing further ion channels to be modulated. This process is closely coupled by feedback. Therefore the waveform produced at the biological cell varies depending on parameters such as the ion channels or receptors which are modulated and the length of time that those ion channels or receptors are activated or inhibited.

Compounds that affect waveforms produced at biological cells may be useful in treating or ameliorating a range of diseases and disorders. For example, action potentials control the function of nerve and muscle tissue, and accordingly influence many physiological functions including the capacity of a body to influence pathology. Similarly, other waveforms such as synaptic events are involved in many nervous system processes. Compounds that affect the production of waveforms at biological cells may therefore be useful in the treatment or amelioration of, for example, a range of neuromuscular, cardiac, pain, affective and cognitive disorders.

However, the effect of any particular compound on a waveform is difficult to assess. As the production of a waveform in a cell involves individual contributions from multiple ion channel or receptor types, the duration of each waveform, the peak membrane potential and many other parameters may vary. Therefore, all necessary ion channel or receptor types to produce a waveform must be present and functional in order to properly observe the effects of the compound on the biological cell. This is usually performed by observing effects of compounds in intact samples of biological tissue, such as recording action potentials in nerve fibres in a living animal model or recording cardiac action potentials by isolation of a purkinje fibre from a dog heart. The requirement for biological tissue limits the number of compounds that can be assessed in a given period of time.

One method for determining the effects of a compound on an ion channel is the patch clamp technique. This employs an amplifier, which is connected to a biological cell via an electrode, to hold current (current clamp mode) or voltage (voltage clamp mode) constant at the membrane. For example, when current is held constant, voltage is recorded. However, such methods do not allow changes in a waveform to be monitored.

In particular, the cell attached or excised patch clamp technique allows the determination of the effect of a compound on a specific ion channel or receptor type of interest. This technique comprises an electrode which is attached to a patch of membrane of a biological cell around an ion channel or receptor of interest. A compound may then be applied to the inner or outer surface of the patch of membrane and the activity of that ion channel or receptor, as acted upon by the compound, measured. However, this process requires the harvesting of many cells to ascertain the effects of the compound on different ion channels or receptors and only determines the action of the compound on that specific ion channel or receptor without the reciprocal influence of the other ion channels or receptors.

Other patch clamp methods, such as the whole cell technique, allow analysis of the electrophysiology of an entire cell. Tests using these methods require many parameters to be simultaneously monitored, which greatly complicates the acquisition and analysis of results. These experimental difficulties mean that in many cases it takes a substantial amount of time to determine exactly how a compound is affecting the cell; it is much more difficult and time consuming to confidently determine on which ion channel or receptor type a compound acts.

Consequently, as waveforms are produced by a number of ion channel or receptor types in a biological cell, it has been difficult to determine the effect of a compound at only one of the ion channel or receptor types involved in producing the waveform. As all of the ion channel or receptor types involved in producing the waveform must be functional, the addition of a compound to this system may modulate any one or more of the ion channel or receptor types involved.

Conversely, it has been possible to determine if a compound binds to, for example a sodium channel, by directly measuring the binding at that channel. However, a large number of changes occur at, for example, sodium channels when they are activated and it is difficult to predict the effect that these channels have on other ion channels when they are assayed in isolation. Consequently, it has been difficult to determine the effect that modulation of an ion channel or receptor will have on the waveform that the ion channel or receptor produces.

### SUMMARY OF THE INVENTION

The present invention is based on the surprising finding that a dynamic clamp can be used to determine the activity of compounds at one or more ion channel or receptor types that are involved in providing a waveform in a biological cell.

Accordingly, in one aspect the present invention provides a method of assaying a compound for its ability to modulate an ion channel or receptor type, the method comprising:
a) providing a dynamic clamp in electrical contact with a biological cell (or part thereof) in which one or more ion channel or receptor types for providing a waveform are functional and in which one or more ion channel or receptor types for providing a waveform are either not present or not functional;
b) causing the dynamic clamp to apply a signal simulating the function of at least one of the one or more ion channel or receptor types that are either not present or not functional in the biological cell (or part thereof) based on modulation of the ion channel or receptor types that are functional in the biological cell (or part thereof) to thereby provide the waveform at the biological cell (or part thereof);
c) exposing at least one of the one or more functional ion channel or receptor types to a compound; and
d) detecting modulation of the waveform at the biological cell (or part thereof), wherein modulation of the waveform is indicative of a compound that modulates the at least one functional ion channel or receptor types.

The dynamic clamp advantageously simulates the function of one or more ion channel or receptor types that are either not present or functional in the biological cell (or part thereof). This means that the assay may only involve a limited number of ion channel or receptor types in a biological cell, allowing assays to be conducted that provide a greater amount of information about the effect of the compound on the ion channel or receptor type that is modulated. Furthermore, the assay also illustrates the effect that modulation of the ion channel or receptor type may have on waveforms produced.

In another aspect, the present invention provides an apparatus for performing the method of the invention.

In a further aspect, the present invention provides an apparatus for assaying a compound's ability to modulate an ion channel or receptor type in a biological cell (or part thereof), the apparatus including:
a) One or more electrodes adapted to be provided in electrical contact with the biological cell (or part thereof), wherein the one or more electrodes are configured:
   i. to detect modulation of one or more functional ion channels or receptor types for providing a waveform at the biological cell (or part thereof) and to provide a first signal based on the detected modulation; and
   ii. to apply a second signal to the biological cell (or part thereof);
b) A simulator to simulate the function of at least one or more ion channel or receptor types for providing a waveform that are either not present or not functional in the biological cell (or part thereof);
   i. wherein the simulator is configured to receive the first signal from the one or more electrodes and to provide the second signal to the one or more electrodes;
   ii. wherein the second signal simulates the function of at least one of the one or more ion channel or receptor types that are either not present or not functional based on the first signal, to thereby provide the waveform at the biological cell (or part thereof).

In another aspect, the present invention provides an apparatus for assaying a compound for its ability to modulate an ion channel or receptor type, the apparatus including:
(a) One or more electrodes to measure an electrophysiological parameter at a biological cell (or part thereof) and to control a current or voltage applied to the biological cell (or part thereof), wherein the one or more electrodes are adapted for electrical connection with the biological cell (or part thereof);
(b) One or more amplifiers to assist in measuring the electrophysiological parameter at the biological cell (or part thereof) and to assist in controlling the current or voltage applied to the biological cell (or part thereof), wherein the one or more amplifiers are electrically connected to the one or more electrodes; and
(c) Software to simulate the function of one or more ion channel or receptor types in a biological cell (or part thereof), which function is simulated by receiving the measurement of the electrophysiological parameter at the biological cell (or part thereof) from the one or more amplifiers, determining the current or voltage to be applied to the biological cell (or part thereof) based on said measurement, and transmitting an electrical signal to the one or more amplifiers to control the current or voltage applied to the biological cell (or part thereof).

In another aspect, the present invention provides a process, including:
receiving data detected from the modulation of at least one ion channel or receptor type at a biological cell (or part thereof);
processing the data to determine a signal to be applied to the biological cell (or part thereof), wherein the signal represents one or more ion channel or receptor types that are either not functional or not present in the biological cell (or part thereof); and
applying the signal to the biological cell (or part thereof).

In further aspects, the present invention also provides a computer-readable storage medium having stored thereon programming instructions for performing the above process, and a system configured to perform the above process.

For a better understanding of the invention and to show how it may be performed, an embodiment of the invention is further described by way of non-limiting example, by reference to the accompanying drawings, in which:

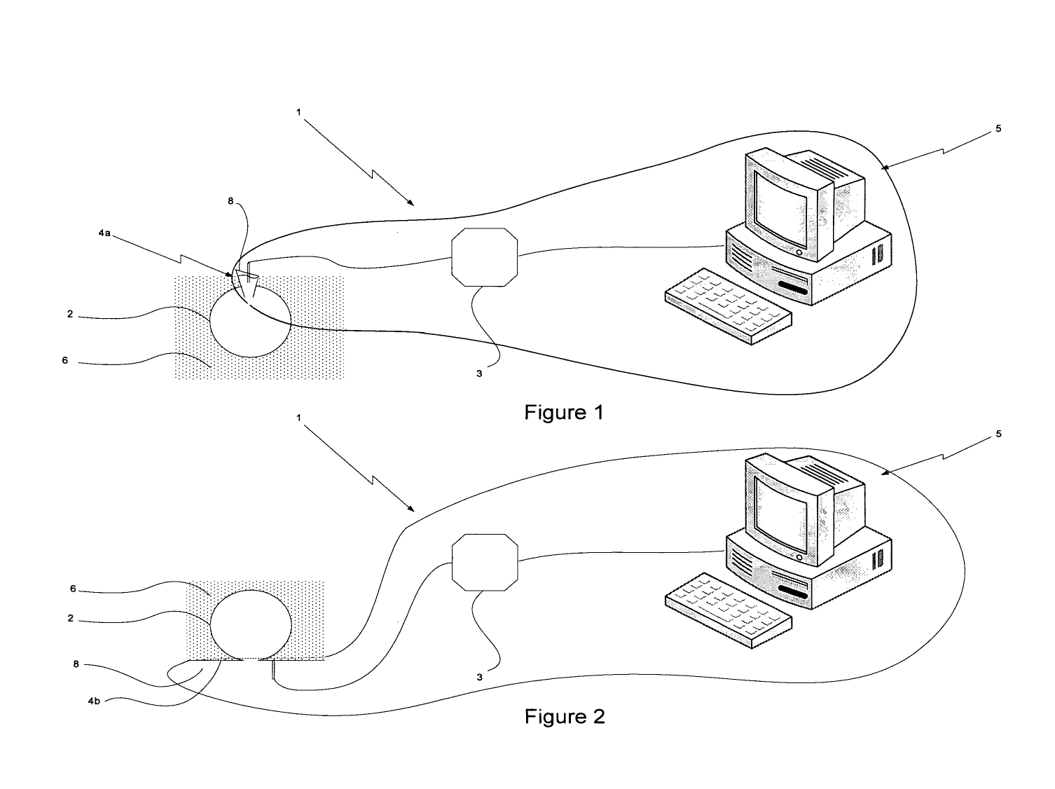

FIG. 1 shows a pipette patch clamp system for the measurement of waveforms, in accordance with an embodiment of the present invention.

FIG. 2 shows a planar patch clamp system for the measurement of waveforms, in accordance with an embodiment of the present invention.

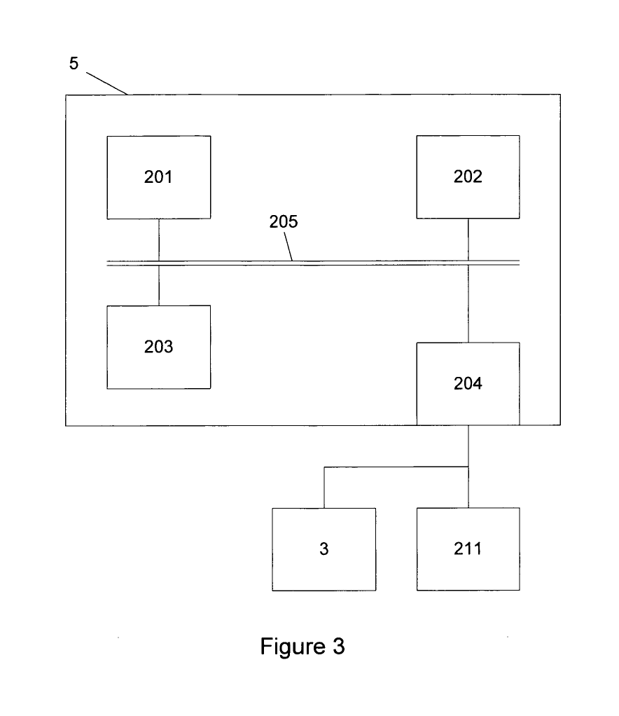

FIG. 3 is an example computing system that may be used in accordance with an embodiment of the present invention.

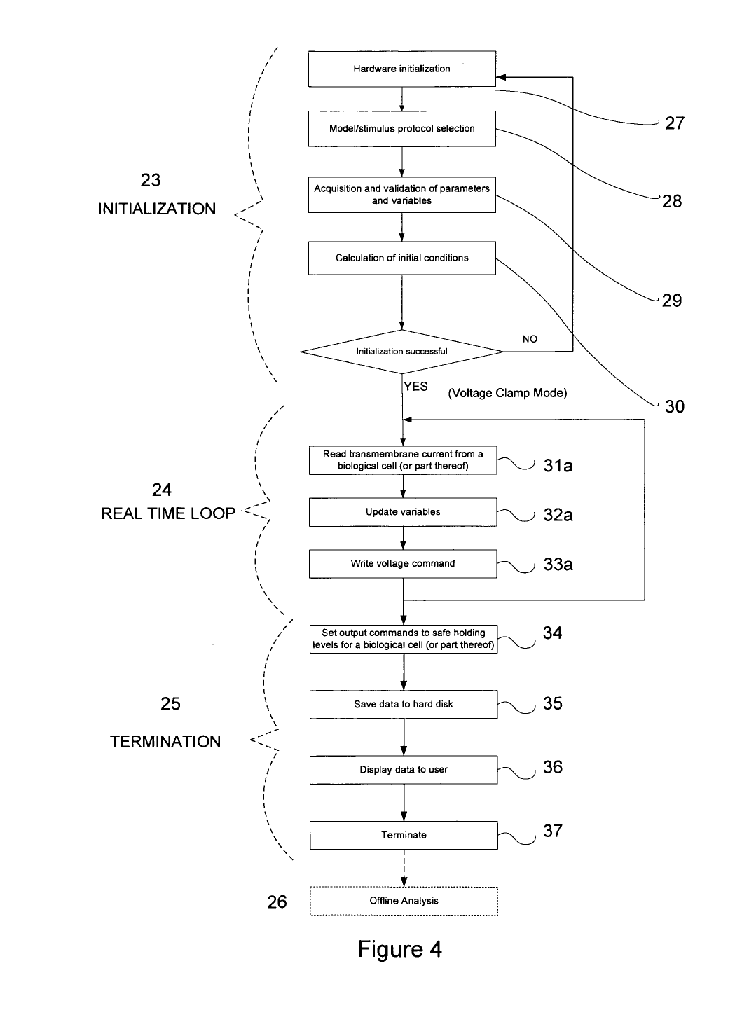

FIG. 4 is a flow chart of a computer program operating in voltage clamp mode in accordance with an embodiment of the present invention.

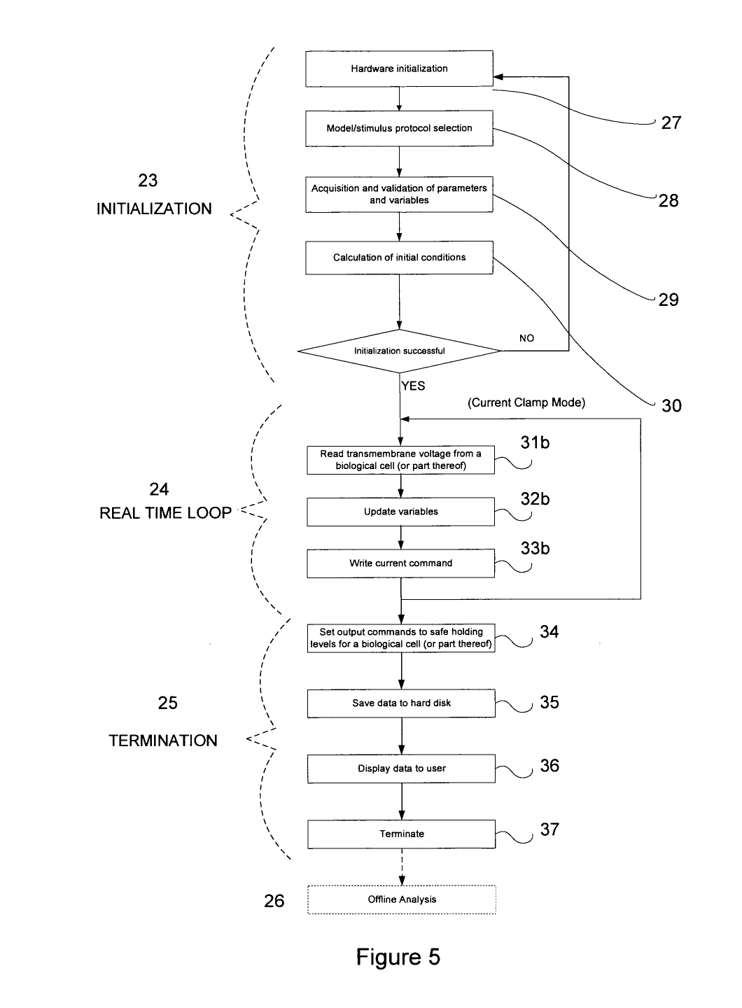

FIG. 5 is a flow chart of a computer program operating in current clamp mode in accordance with an embodiment of the present invention.

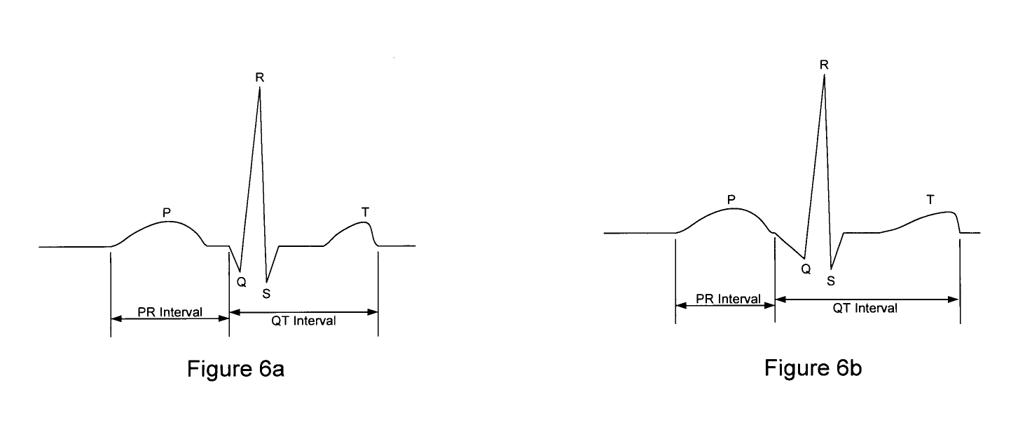

FIGS. 6a and 6b are exemplary electrocardiogram outputs, the output of FIG. 6b showing an elongated QT interval.

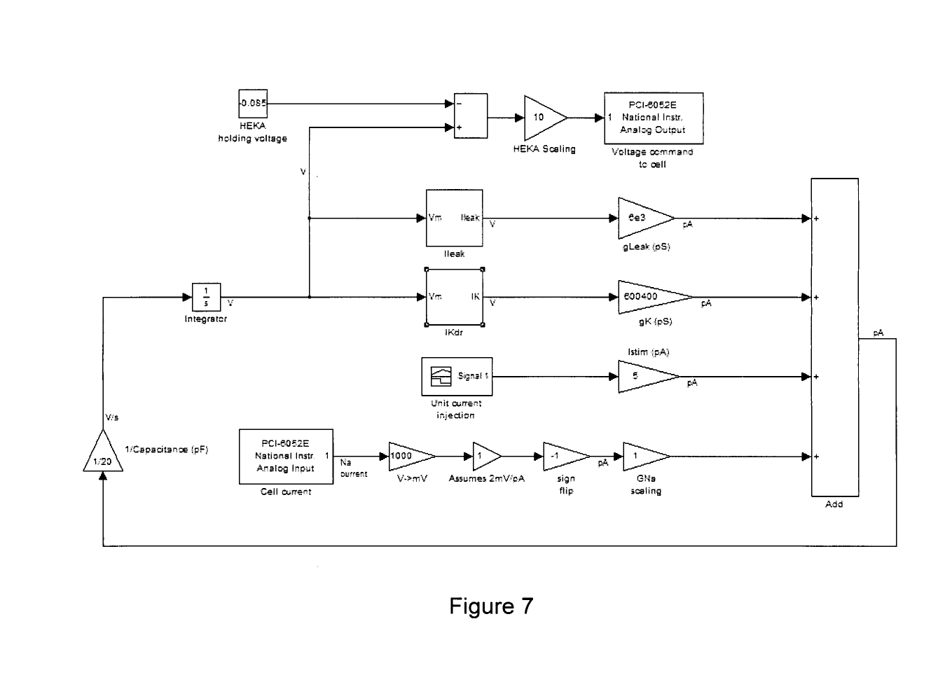

FIG. 7 is a diagram of a dynamic clamp system used in accordance with an embodiment of the present invention.

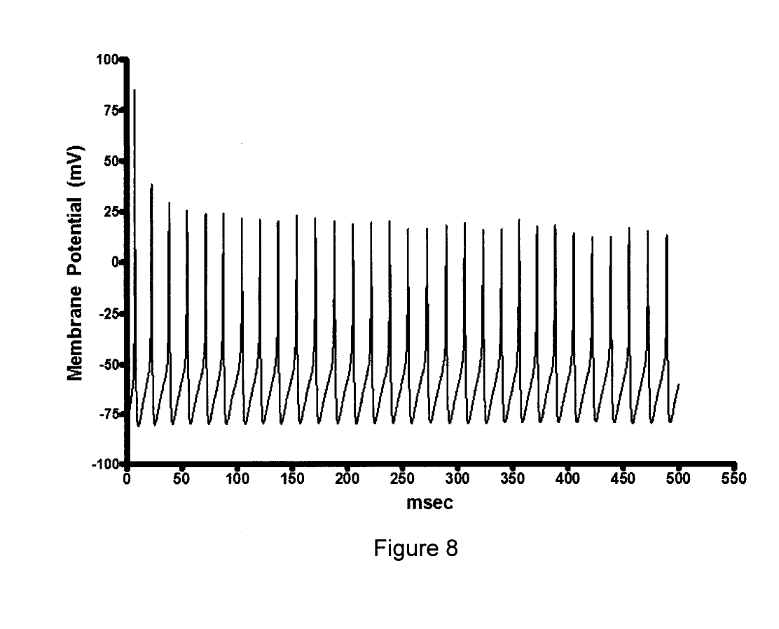

FIG. 8 illustrates a steady state action potential firing of 50-100 Hz at HEK cells controlled by a dynamic clamp system, in which the cells express Naᵥ1.4 sodium channels.

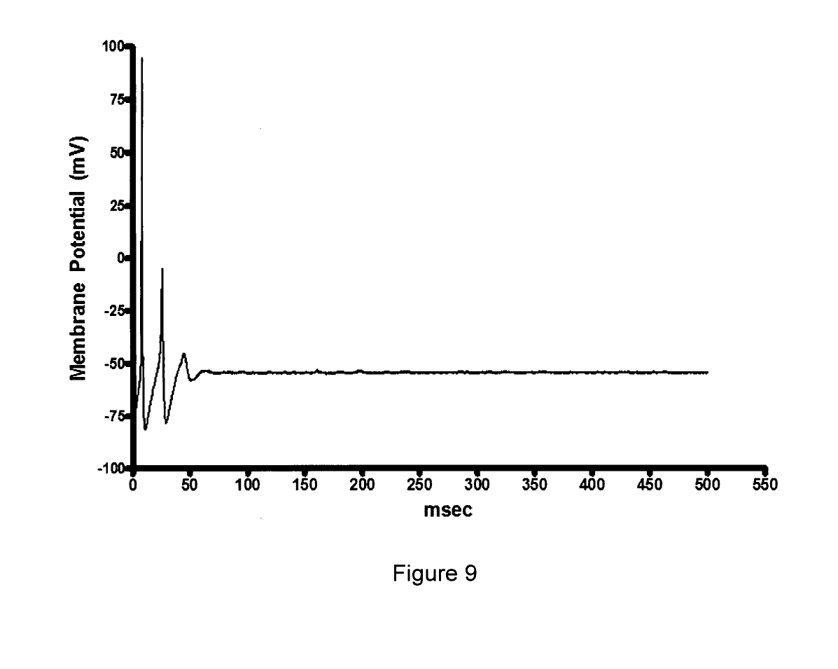

FIG. 9 illustrates the decrease in action potential firing rate achieved when carbamazepine is perfused onto HEK cells controlled by a dynamic clamp system, in which the cells express Naᵥ1.4 sodium channels.

Like features will hereinafter be referred to with like numbers.

### DETAILED DESCRIPTION OF AN EMBODIMENT OF THE INVENTION

A dynamic clamp detects an electrophysiological parameter (which may, for example, include current, voltage or capacitance) of a biological cell (or part thereof), and then applies a signal (for example, voltage or current) to the biological cell (or part thereof) to achieve a desired effect on the electrophysiological parameter. The step of applying the signal to the biological cell (or part thereof) requires the calculation of the amount of, for example, the voltage or current that must be applied to the cell (or part thereof) to produce the desired effect. Following the detection of an electrophysiological parameter and the subsequent application of the signal to the biological cell (or part thereof), the dynamic clamp continually repeats the process.

In an embodiment of the present invention, a dynamic clamp 1 is provided in electrical contact with a biological cell 2, as shown in FIGS. 1 and 2. In assaying a compound for its ability to modulate an ion channel or receptor type, the dynamic clamp assists in providing a waveform at a biological cell (or part thereof).

As used herein, the term “waveform” would be understood by a person skilled in the art, and includes any variation (for example variations in the amplitude or frequency) in an electrophysiological parameter (for example the trans-membrane voltage) over time at a cell. Such variations result from modulation of a number of ion channel or receptor types at the cell. In one embodiment, the waveform is an action potential or synaptic event. In another embodiment, the waveform is an action potential.

A waveform at a biological cell (or part thereof) is generally produced by virtue of a functional inter-relationship between a number of different types of ion channels or receptors. Modulation of one, or a group of ion channels or receptors results in electrophysiological changes at the membrane of the cell, causing further ion channels to be modulated, resulting in a waveform. Ion channels including, for example, sodium channels, potassium channels, calcium channels, chloride channels and hyperpolarisation-activated cation channels may involved.

Advantageously, in the present invention it is only necessary for one of the ion channels or receptor types to be present in the biological cell (or part thereof). The function of the remaining ion channels or receptor types which are required to provide a waveform may be simulated using a dynamic clamp, which is configured to provide a real time feedback loop with the ion channels or receptor types that are present. To achieve this, the dynamic clamp can apply a signal to the cell or part thereof. The signal is used to represent the electrophysiological changes to the cell that would be induced by the remaining ion channels. This allows the effects of a compound at only one type of ion channel or receptor to be detected, while also observing the effect of the compound on the waveform of a more complex system.

This is particularly important as the effect of a compound on an ion channel or receptor involved in producing a waveform may affect parameters such as the frequency of waveform generation, and the morphology of the waveform generated. For example, the morphology of an action potential includes the half width, rise time, decay time, time between successive action potentials and rebound voltage. The assay according to the present invention may measure one, a number, or all of these changes.

The method of the present invention therefore provides a phenotypic screen that provides high content information on waveform properties and is rapid enough for the drug discovery cycle.

In one embodiment, the dynamic clamp applies a voltage signal to the biological cell (or part thereof), and modulation of the waveform at the biological cell (or part thereof) is detected by measuring a current signal at the biological cell (or part thereof). In this embodiment the voltage is clamped.

To simulate a particular voltage, the dynamic clamp may measure the membrane current of a biological cell (or part thereof), and use this parameter to determine the amount of voltage to be applied to the cell (or part thereof). If there is insufficient current to produce a waveform, then the dynamic clamp may modulate the amount of current applied by mathematical scaling in the feedback system.

In another embodiment, the dynamic clamp applies a current signal to the biological cell (or part thereof), and modulation of the waveform at the biological cell (or part thereof) is detected by measuring a voltage signal at the biological cell (or part thereof). In this embodiment the current is clamped.

To simulate a particular conductance, the dynamic clamp may use the measured membrane potential of a biological cell (or part thereof) and the reversal potential for that conductance (the membrane potential at which there is no net flow of ions from one side of the membrane to the other) to determine the amount of current to be applied to the cell (or part thereof).

If there is insufficient current to produce a waveform, then a capacitive current term may be used to control the apparent capacitance of the cell (or part thereof) and in this way provide a precise control on the ratio of conductance to capacitance. The capacitive current term is calculated by measuring the rate of change of the voltage, and its application may decrease the apparent capacitance of the biological cell (or part thereof) to compensate for the lack of current.

The dynamic clamp may also be used to account for leak conductance at the cell (or part thereof). Leak conductance may occur because ion channels or receptors in the cell (or part thereof) are open, allowing the passage of ions. If the dynamic clamp does not account for leak conductance, then the assay results may be affected.

The dynamic clamp may also be used to account for and subtract the signal arising from one type of ion channels or receptors involved in the production of a waveform at the biological cell (or part thereof). For example, the signal arising from one type of ion channels or receptor can be removed using a dynamic clamp to provide further information on the effect of that ion channel or receptor on the waveform. Such techniques are known to a person skilled in the art and are discussed for example in Prinz et al., (2004) Trends in Neurosciences, 27, 218-224.

Many types of dynamic clamp may be used in the method according to the present invention. As shown in FIGS. 1 and 2, the dynamic clamp 1 may include, but is not limited to, one or more electrodes 4, and a simulator. The simulator may include an amplifier 3, and computational software, which may be stored on and executed by a computing system 5.

In one embodiment, the one or more electrodes in contact with the biological cell (or part thereof) are sharp electrodes. A sharp electrode is a type of micropipette that has a very fine pore that allows slow movement (generally only capillary action) of solution through the electrode, thereby providing a minimal effect on the composition of the intracellular fluid. In use, a sharp electrode punctures the cell membrane so that the tip of the electrode is inside the cell.

In another embodiment, the one or more electrodes in contact with the biological cell (or part thereof) are patch electrodes. A patch electrode comprises a much larger pore than a sharp electrode. For a patch electrode, a high resistance (typically hundreds of megaohms to several gigaohms) electrical seal is formed between the electrode and the membrane of a biological cell. The membrane of the biological cell is then ruptured (such as by suction) so that a solution in a pipette (for pipette patch electrodes) or adjoining the aperture (for a planar patch electrode) is able to mix with the intracellular fluid. This is also known as a whole cell patch and allows an electrophysiological parameter across an entire cell membrane to be measured.

In one embodiment, a pipette patch electrode 4a (FIG. 1) involves the formation of a high resistance electrical seal between a micropipette (the electrode) and a membrane of the biological cell 2. Once the seal is formed, a solution 8 in the micropipette is able to mix with the intracellular fluid.

In contrast, a planar patch electrode 4b (FIG. 2) may involve the formation of a high resistance electrical seal between an aperture of a usually flat substrate (the electrode) and a membrane of the biological cell 2. In general, a well is provided at each aperture of the substrate, and after a seal is formed and the membrane ruptured, a solution 8 in this well is able to mix with the intracellular fluid.

As the planar electrode may comprise multiple apertures at which high resistance electrical seals may be formed with different cells, planar patch electrodes are generally more adaptable to high throughput, automated screening techniques. For example, electrodes which accommodate 16, 48, 96 or 384 cells for simultaneous recordings may be employed. Such electrodes could be, or would be similar to the QPlate (Sophion Bioscience) or PatchPlate PPC and PatchPlate substrates (MDS Analytical Technologies) or those used for the Patchliner and Synchropatch systems (Nanion Technologies GmbH) or the IonFlux system (Fluxion Biosciences).

Regardless of the type of patch electrode, it is important to achieve a high resistance electrical seal between the electrode and the membrane of the biological cell (or part thereof). If the seal is of poor quality, then assay results may be affected.

Many of the types of electrodes discussed above require the use of a solution 8 which is in contact with the intracellular fluid of the cell. The composition of the solution used with the electrode depends on the assay to be conducted, and a person skilled in the art would be able to select a suitable solution without undue experiment. If the solution is to be able to mix with the intracellular fluid, the solution generally comprises a high concentration of electrolytes and is iso-osmotic to the intracellular fluid. When conducting assays with patch electrodes, this solution may be changed or altered. For example, in one embodiment the concentration of compound to be tested in the solution may be altered, allowing a dose-response curve to be determined.

The dynamic clamp may comprise one or more electrodes 4. In one embodiment, the dynamic clamp comprises two electrodes which are in contact with a biological cell (or part thereof). In another embodiment, the dynamic clamp comprises one electrode which is in contact with a biological cell (or part thereof).

These electrodes may provide a continuous clamp, a discontinuous clamp or a two electrode clamp. A continuous clamp comprises one electrode, and that electrode simultaneously and continuously detects an electrophysiological parameter and applies the signal (such as the voltage or current) to a cell (or part thereof). In contrast, a discontinuous clamp also comprises one electrode, but that electrode switches between detecting an electrophysiological parameter and applying the signal to the cell (or part thereof). In a two electrode clamp there are two electrodes: one electrode detects an electrophysiological parameter and the other applies the signal to the cell (or part thereof).

The dynamic clamp may also comprise a ground electrode. A ground electrode sets the ground reference point for electrophysiological measurements. The ground electrode may be in contact with a bath solution surrounding the biological cell (or part thereof). In one embodiment the ground electrode is a silver chloride coated silver wire. In another embodiment the ground electrode is a platinum electrode. The ground electrode may also be coated with agar.

The bath solution 6 selected may depend on a number of factors including, for example, the experiments to be conducted and the type of cell used. An appropriate bath solution 6 may be selected by a person skilled in the art without undue experiment.

Other current and voltage clamp systems that may be adapted for use in the method according to the present invention are described in The Axon Guide: A Guide to Electrophysiology and Biophysics Laboratory Techniques, MDS Analytical Technologies, 2008.

In addition to the one or more electrodes, the dynamic clamp also comprises a simulator to simulate the function of at least one or more ion channel or receptor types for providing a waveform that are either not present or not functional in the biological cell (or part thereof). The simulator is configured to receive a first signal from the electrode, which is based on the detected modulation of the ion channel or receptor, and to provide a second signal to the electrode to be applied to the cell (or part thereof). The signal provided to the cell simulates the function of at least one or more of the ion channel or receptor types that are either not present or not functional based on the first signal, to thereby provide the waveform at the biological cell (or part thereof).

The simulator may also include an output to display at least one of a waveform or other data to allow a compound's ability to modulate an ion channel or receptor type to be determined. In this embodiment, the other data displayed by the software may include, for example, the raw data obtained from the assay, or an icon or symbol that indicates whether or not there has been any change in the output following administration of the compound to the biological cell (or part thereof).

In another embodiment, the simulator comprises one or more amplifiers. The simulator may also comprise a suitably programmed computing system. In a further embodiment, the computing system operates to control the amplifier to provide the second signal to the one or more electrodes, and the computing system operates to receive the first signal from the one or more electrodes. The computing system may also operate to analyse the first signal and control the amplifier in accordance with analysis of the first signal.

In one embodiment, the dynamic clamp comprises one or more amplifiers, as shown for example as 3 in FIGS. 1 and 2. Many amplifiers may be used to assist in the measurement of an electrophysiological parameter at the biological cell (or part thereof), and to also assist in the control of the signal applied to that cell (or part thereof). However, in another embodiment, separate amplifiers may be used to perform these two functions.

The type, or characteristics (for example input impedance or bandwidth), of the amplifier required will vary depending upon a number of factors including, but not limited to, the type of electrode used (for example sharp electrode or patch electrode) and if the electrodes provide a continuous clamp, a discontinuous clamp or a two electrode clamp. The amplifier may also provide features such as series resistance compensation, capacitance compensation, low-pass filters, Bridge Balance and features to assist in record keeping, cell penetration and patch rupture. The amplifier may also comprise a feedback amplification system to further control the current when using a patch clamp in current clamp mode (a patch clamp in voltage clamp mode does not require such a feedback amplification system).

For example, when performing patch electrode assays, suitable amplifiers may include the EPC10 (HEKA Elektronik), the Axopatch 200B (Molecular Devices), the VE-2 (Alembic Instruments Inc.) and the MultiClamp 700A (Molecular Devices). When performing sharp electrode experiments, the Axoclamp 2B (Molecular Devices) may be a suitable amplifier. A person skilled in the art would be able to select an appropriate amplifier without undue experiment.

The dynamic clamp may also comprise computational software, which may be stored at a computing system 5 or other similar processing device. The computing system 5 is typically adapted to receive signals indicative of electrophysiological parameters, perform processing of the parameters and control the signal application to the cell. Accordingly, any suitable form of computing system can be used.

An example computing system is shown in FIG. 3. In this example, the computing system 5 includes a processor 201, a memory 202, an input/output device 203, such as a keyboard and display or the like, and an external interface 204, coupled together via a bus 205. In use, the external interface 204 may be coupled to a remote store, such as a database 211, as well as to the amplifier 3.

In use, the processor 201 executes software stored in the memory 202. The software defines instructions, typically in the form of commands, which cause the processor 201 to perform the steps outlined above, and described in more detail below, to control the dynamic clamp while performing the assay. The software may also display results to allow the outcome of the assay to be determined. Accordingly, the computing system 200 may be any form of processing system, such as a computer server, a network server, a web server, a desktop computer, a lap-top or the like. Alternative specialised hardware may be used, such as FPGA (field programmable gate array), or the like.

In one embodiment, the computing system is used to detect modulation of the waveform at the biological cell (or part thereof) (which is indicative of a compound that modulates at least one type of functional ion channel or receptor in the cell (or part thereof)).

The computing system may also determine the signal that should be provided to the biological cell (or part thereof) to simulate the function of one or more ion channel or receptor types that are either not functional or not present in the biological cell (or part thereof). The amount of voltage or current to be provided to the cell (or part thereof) is determined based on modulation of the ion channels or receptors that are functional in the biological cell, as measured by electrophysiological measurements of that cell (or part thereof). This assists in understanding the effect that modulation of a type of functional ion channel or receptor in a biological cell (or part thereof) by a compound will have on the waveform.

The simulated signal is generated by modelling data representative of the absent types of ion channels or receptors, which modelling preferably occurs in software. The data for the model can be either collected by recording the action of those types of ion channels or receptors or by input of known data. As the data are representative of the conductance of ions across a cell membrane during a waveform, the data will normally be stored in the form of mathematical descriptions of virtual conductances (simulation algorithms) in either the memory 202 or database 211. In this manner, the software can model either components of a biological cell or the entirety of a biological cell.

The simulation algorithms are designed to self-adjust to account for changes in the cell. The complexity of the simulation algorithms depends upon the number of factors that the dynamic clamp is designed to account for, including the number of ion channels or receptor types to be simulated. For example, for skeletal muscle cells the action potential produced largely arises from the interaction between sodium channels and potassium channels. However, for cardiac muscle cells the action potential produced arises from the interaction of a greater number of ion channels or receptor types, resulting in more complex algorithms.

In addition, the data may contain parameters to account for losses in hardware, losses in the electrolyte in the pipette electrode (if used), at least one stimulation protocol and calculated variables as hereafter discussed. Accordingly, the simulation takes the measured waveform of the biological cell (or part thereof) and generates a signal representative of the absent types of ion channels or receptors, to encourage the waveform to develop as it would if the absent types of ion channels and receptors were functional.

The model of virtual conductances may include:
the kinetics of the virtual conductance (the rates of change of conductance to particular stimuli);
the voltage dependence of virtual conductances (the equilibrium open probability of a conductance);
the maximum conductance of the biological channel expressed in the cell that is being recorded. This is particularly useful in determining a scaling factor for voltage clamp methods as this defines the maximum conductance that the channels expressed in the cell (or part thereof) will produce. Moreover, without such scaling there may be insufficient current to support waveform, and especially action potential, generation. Scaling may also be useful for increasing reproducibility of the assay as variables such as membrane capacitance, leak conductance and maximum conductance of the expressed channel can all be scaled to predefined ratios;
the electrochemical properties of the system, including the reversal potentials of the virtual conductances (the membrane potential at which there is no net transmembrane flow of ions for a particular conductance); and
other passive properties of the model system, including passive properties of both the biological cell (or part thereof) and the components or entirety of the virtual cell. This may include the desired capacitance and resting conditions (such as resting conductance and resting voltage).

The stimulation protocol is a user defined signal applied to the biological cell (or part thereof) to generate desired physiological responses in the biological cell (or part thereof). In the present case, the desired physiological response is a waveform such as an action potential. These stimulation protocols allow the user to determine how the cell (or part thereof) will be stimulated and to what degree. For example, these protocols allow the user to determine whether the cell (or part thereof) is to be stimulated using voltage or current and the levels at which these stimuli will be set.

Stimulation protocols are useful where a biological cell (or part thereof) is in a state whereby a waveform will not be produced, or will not be produced repetitively. When a biological cell (or part thereof) is in such a state, assaying compounds may not be possible as the modulation of a waveform cannot be observed if no waveform is produced, or if it is produced too irregularly or too few times to allow accurate results to be measured. In such circumstances, the stimulation protocol can be used to produce a waveform, or cause its repetition. It achieves this by providing a stimulus that would not normally be exhibited by any of the types of ion channels or receptors the function of which the simulated signal is intended to replicate.

As biological cells differ in their electrophysiological properties, calculated variables are included in the simulation to allow the simulated signal to be tailored to the biological cell (or part thereof) to which the compounds to be assayed are exposed. The calculated variables include the capacitance of the biological cell (or part thereof) (determined from electrode measurements), modified virtual conductances (which are updated according to the cell (or part thereof) to which the apparatus is in contact and modelled to form the simulation algorithms), and an output command signal that is dependent on the mode in which the software is operating (i.e. voltage or current-clamp mode).

In the voltage-clamp mode, the transmembrane or ionic current is measured by the amplifier through the electrode. It is then scaled to match the electrical parameters of the model system. The simulated signal, or transmembrane voltage (membrane potential), is then calculated by collecting the contributions from each of the virtual conductances, the capacitance of the virtual cell, the scaled ionic current recorded from the biological cell (or part thereof) and the selected stimulation protocol. The output command signal is then set to this transmembrane voltage and subsequently sent to an amplifier for application to the biological cell (or part thereof).

In the current-clamp mode, the transmembrane voltage of the biological cell is measured by the amplifier through the electrode. The measurement may be filtered and sent to the computing system. The filtration prevents amplification of noise that could affect the calculation of the capacitance compensation term as previously described. The software calculates the capacitance compensation term by determining the capacitance of the cell (or part thereof) and then applying a scaling factor to the rate of current application from each of the virtual conductances and the stimulation protocol. This can mathematically compensate for natural differences in the total capacitances of cells and normalise to a predefined capacitance level across all cells. The scaled output command signal is then sent to the amplifier for application to the biological cell (or part thereof).

The software may be stored on any computer-readable medium such as a hard disk, removable memory device, external hard drive etc. In addition, the software may only contain those parameters, stimulation protocols etc that are relevant to performing the task to which the apparatus, interacting with the biological cell (or part thereof), is put.

In order to take readings, the present system passes through a plurality of operational phases as illustrated in FIGS. 4 and 5. These phases optionally include, but are not limited to, initialization 23, real time looping for current or voltage-clamp mode 24, termination 25 and offline analysis 26.

The initialization phase 23, consists of hardware initialization 27, stimulation protocol selection 28 (for the reasons discussed earlier), acquisition and validation of parameters and variables 29, and calculation of initial conditions 30.

In particular, the hardware is initialized and tested to ensure it is functioning properly. This part of the initialization phase may include the testing of the operational limits of the hardware; passing inputs, to which inputs there is a predetermined or expected system response, to the hardware and comparing the hardware response to the predetermined response; and so forth.

The acquisition and validation of parameters and variables is particularly important so as to ensure all data necessary for the accurate simulation of responses to measurements taken from the biological cell (or part thereof), can be produced. If some data is missing, such as a parameter representative of the response of a functional ion channel or receptor type that is not present or not functional in the biological cell, it may be collected before testing commences. This step may also ensure that the correct data for the operating mode of the apparatus, and the selected stimulation protocol, is acquired. It should be noted that although the system can operate in both current and voltage-clamp modes, the parameters and variables appropriate to one mode of operation may not be appropriate for the other.

The last stage of initialization is the calculation of initial conditions. This process sets the equipment default and references values which are useful in the process of recording data, such as a reference voltage and current. In addition, this step allows the calculated variables to be determined in order to adapt the test to different biological cells (or parts thereof) and cells that have been intentionally experimentally modified (i.e. by administration of other compounds to simulate a condition the present compound is being developed to treat).

The next phase in the program is the real time looping phase 24. If the apparatus is operating in voltage-clamp mode, the transmembrane current from the biological cell (or part thereof) is measured 31a (FIG. 4). The variables stored in software are updated in accordance with the measurement 32a and an output command is generated. Simultaneously, this output command, that can be representative of the restoration current (the current required to return the membrane potential of the biological cell (or part thereof) to the resting potential), or is alternatively the ionic currents that would be exhibited by functional ion channel and receptor types that are either not present or not functional in the biological cell (or part thereof), is written to memory 33a.

Similarly, when the apparatus is operating in current-clamp mode, the transmembrane voltage is measured by the amplifier through the electrode 31b (FIG. 5). The variables stored in software are updated in accordance with the measurement 32b and an output command is generated. Simultaneously, this output command is written to memory 33b.

During the termination phase 25, the output commands are set to levels at which it is safe to hold the biological cell (or part thereof) 34 (FIGS. 4 and 5). This ensures the cell remains functional, without being damaged, that parameters against which measurements are taken and responses are generated remain fixed and that the cell is in a predictable state for the next experiment.

The data is then saved to hard disk or other appropriate medium 35, displayed to the user if desired 36, and the process is terminated 37.

Finally, during the offline analysis phase 26, calculations are performed to identify the initial conditions and parameters appropriate for the next iteration of testing. This data may also be displayed to the user. If a sufficient number of experiments have been performed at, for example, the various concentrations of compound, a model can be fitted to the data to describe the action of the compound on the system.

The program may be stored in a single place on a computer readable medium. However, it may be advantageous for individual devices to store data relevant to their own operation. For example, the amplifier may store its own initialization data and sequence for initializing, and the computing system may store data for applying tests to determine the responses generated by the software are appropriate.

The production of a waveform involves the activation of large numbers of multiple types of ion channels or receptors. Accordingly, it is possible to produce a waveform in a whole biological cell or in a part of a biological cell. In one embodiment, a whole biological cell is used.

In another embodiment, part of a biological cell is used. For example, the waveform may be produced at a part of a biological cell using a macropatch. A macropatch employs a large diameter pipette (for a pipette patch electrode) or a large aperture electrode (for a planar patch electrode) to surround a number of ion channels or receptors on a cell membrane. After forming a seal on the cell membrane using the macropatch, the electrode may be quickly withdrawn to separate a portion of the cell membrane (an inside-out patch). Alternatively after forming a seal, the cell membrane inside the electrode may be ruptured and then the electrode slowly withdrawn to separate a portion of the cell membrane (an outside-out patch).

In the method according to the present invention, a waveform is provided at the biological cell (or part thereof), and the effect of the compound at a functional ion channel or receptor type is determined by detecting modulation of the waveform at the biological cell (or part thereof).

A waveform may be provided in the biological cell (or part thereof) in a number of ways. For example, in one embodiment the waveform may be initiated by the dynamic clamp. In another embodiment, the waveform may be initiated by the action of a compound at the one or more ion channel or receptor types that are functional in the biological cell (or part thereof).

At least one or more functional ion channel or receptor types may be exposed to a compound in a number of ways. For example, a compound may be applied to a bath solution which surrounds the biological cell (or part thereof). In another embodiment, the compound may be administered to the inside of the cell (or part thereof) through a recording pipette or recording aperture (in the case of a planar electrode) which is in contact with the inside of the cell (or part thereof).

The compound may modulate an ion channel or receptor by contacting that ion channel or receptor on the outside of the cell, or on the inside of the cell. Some compounds will not be able to pass through the cell membrane and their effect on the cell therefore may be more limited. On the other hand, some compounds will be able to pass through the cell membrane and act intracellularly or extracellularly. Compounds that are able to pass through a cell membrane may be advantageous as this is a desirable characteristic of many pharmaceuticals.

In the biological cell (or part thereof) according to the invention, one or more ion channel or receptor types for providing a waveform are functional, and one or more ion channel or receptor types for providing a waveform are either not present or not functional.

As used herein, the term “functional”, as applied to an ion channel or receptor, means that the ion channel or receptor may be involved in providing a waveform.

In one embodiment, an ion channel or receptor type is present in the biological cell (or part thereof), but that ion channel or receptor type is not functional due to pharmacological inhibition. This may allow a greater number of types of biological cells (or parts thereof) to be used in the assays according to the present invention. For example, tetrodotoxin (TTX), saxitoxin or lidocaine may be used to block most voltage gated sodium channels. In another example, tetraethylammonium (TEA) and 4-aminopyridine (4-AP) may be used to block most voltage gated potassium channels.

In another embodiment, an ion channel or receptor type is present in the biological cell (or part thereof), but the dynamic clamp is used to subtract the signal from that ion channel or receptor type. This may allow validation of the predicted effect of that ion channel or receptor type on the waveform produced at the biological cell (or part thereof), or may provide additional information regarding the behaviour of that ion channel or receptor type in the biological cell (or part thereof). Such techniques are known to a person skilled in the art and are discussed for example in Prinz et al., (2004) Trends in Neurosciences, 27, 218-224. In some cases, the dynamic clamp may also be used to simulate ion channels or receptors that are functional in the biological cell (or part thereof).

The biological cell may therefore be naturally occurring, already in existence, genetically modified or modified by interaction of, for example, an antagonist or virus.

In one embodiment, the one or more ion channel or receptor types for providing a waveform are functional as they are expressed in the biological cell (or part thereof), and the one or more ion channel or receptor types for providing a waveform are either not present or functional as they are not expressed in the biological cell (or part thereof).

Therefore in one embodiment, the biological cell may be a cell in which the genes for the one or more functional ion channel or receptor types have been inserted, or the biological cell may be a cell in which the genes for one or more functional ion channel or receptor types have been removed. In one embodiment, the biological cell is a cell in which the genes for one or more functional ion channel types have been inserted.

To produce a cell expressing one or more ion channels or receptors, the DNA sequence for the ion channel or receptor type may be obtained and then incorporated into an expression vector with an appropriate promoter. Once the expression vector is constructed, it may then be introduced into the appropriate cell line using methods including CaCl₂, CaPO₄, microinjection, electroporation, liposomal transfer, dendrimers, viral transfer or particle mediated gene transfer.

The biological cell line (or host cell) may comprise prokaryote, yeast or higher eukaryote cells. Suitable prokaryotes may include, but are not limited to, eubacteria, such as Gram-negative or Gram-positive organisms, including Enterobacteriaceae. Such Enterobacteriaceae may include Bacilli (e.g. B. subtilis and B. licheniformis), Escherichia (e.g. E. coli), Enterobacter, Erwinia, Klebsiella, Proteus, Pseudomonas (e.g. P. aeruginosa), Salmonella (e.g. Salmonella typhimurium), Serratia (e.g. Serratia marcescens), Shigella, and Streptomyces. Suitable eukaryotic microbes include, but are not limited to, Candida, Kluyveromyces (e.g. K. lactis, K. fragilis, K. bulgaricus, K. wickeramii, K. waltii, K. drosophilarum, K. thermotolerans and K. marxianus), Neurospora crassa, Pichia pastoris, Trichoderma reesia, Saccharomyces cerevisiae, Schizosaccharomyces pombe, Schwanniomyces (e.g. Schwanniomyces occidentalis), and filamentous fungi (e.g. Neurospora, Penicillium, Tolypocladium, and Aspergillus (e.g. A. nidulans and A. niger)) and methylotrophic yeasts (e.g. Hansenula, Candida, Kloeckera, Pichia, Saccharomyces, Torulopsis, and Rhodotorula). Suitable multicellular organisms include, but are not limited to, invertebrate cells (e.g. insect cells including Drosophila and Spodoptera), plant cells, and mammalian cell lines (e.g. Chinese hamster ovary (CHO cells), monkey kidney line, human embryonic kidney line, mouse sertoli cells, human lung cells, human liver cells and mouse mammary tumor cells). An appropriate host cell can be selected without undue experimentation by a person skilled in the art.

In one embodiment, the biological cell (or part thereof) is selected from the group consisting of a human embryonic kidney (HEK) cell, a COS cell, an LTK cell, a Chinese hamster lung cell, or a Chinese hamster ovary (CHO) cell or a Xenopus oocyte. In a further embodiment, the biological cell (or part thereof) is a HEK cell or a COS cell, particularly a HEK 293 cell or a COS-7 cell. In another embodiment, the biological cell (or part thereof) is a HEK cell, particularly a HEK 293 cell.

The type of biological cell selected may affect the dynamic clamping technique employed. For example, the large size of Xenopus oocytes allows a two electrode clamp to be used far more readily than with mammalian cells, which are typically much smaller.

The cell line may then be cultured in conventional nutrient media modified for inducing promoters, selecting transformants, or amplifying the genes encoding the desired sequences. Culture conditions, such as media, temperature, pH, and the like, can be selected without undue experimentation by the person skilled in the art (for general principles, protocols and practical techniques, see Mammalian Cell Biotechnology: A Practical Approach, Butler, M. ed., IRL Press, 1991; Sambrook et al., Molecular Cloning: A Laboratory Manual, Cold Spring Harbor Laboratory Press, 1989). The cells may then be selected and assayed for the expression of the desired ion channel or receptor using standard procedures.

A number of functional ion channels or receptors are involved in providing a waveform in a biological cell. For example, this may include an ion channel selected from the group consisting of a sodium channel, a potassium channel, a calcium channel, a chloride channel or a hyperpolarisation-activated cation channel (H-channel). Accessory subunits of these channels may also be involved in providing a waveform.

As used herein, a receptor for providing a waveform is a receptor that is modulated following contact with a ligand. While modulation of an ion channel may also involve contact with a ligand (ligand-gated ion channels), ion channels may also open and close in response to changes in membrane potential (voltage-gated ion channels), or may be modulated by other means.

As used herein the term “modulating” is used in the broadest sense, encompassing any form or physical or chemical effect. For example, this may include activation or inhibition of the receptor, the effect of agonists or antagonists at the receptor, up-regulation or down-regulation of receptor, inhibition or activation of second messenger molecules or receptor internalisation. In one embodiment, modulation of the ion channel or receptor type is inhibition of the ion channel or receptor type. In another embodiment, modulation of the ion channel or receptor type is activation of the ion channel or receptor type.

Modulation of an ion channel or receptor type also includes modulation of a subunit of the ion channel or receptor type. Selective modulation of specific subunits may be advantageous in the development of compounds with appropriate pharmacological characteristics.

In one embodiment of the invention, the one or more ion channel or receptor types that are functional in the biological cell (or part thereof) are one or more ion channels. In a further embodiment, the one or more ion channel or receptor types that are functional in the biological cell (or part thereof) are one or more voltage-gated ion channels.

The ion channel may be selected from the group consisting of a sodium channel, a potassium channel, a calcium channel, a chloride channel or a hyperpolarisation-activated cation channel. In one embodiment, the ion channel is a sodium channel. In another embodiment, the ion channel is a potassium channel. In a further embodiment, the ion channel is a calcium channel. In another embodiment, the ion channel is a hyperpolarisation-activated cation channel.

Calcium cations and chloride anions are involved in the production of a number of types of waveforms, such as the cardiac action potential and the action potential in various single-celled organisms. Calcium channels are known to play a role in controlling muscle movement as well as neuronal excitation, although intracellular calcium ions can, in some circumstances, activate particular potassium channels. In addition, chloride channels are known to aide in the regulation of pH, organic solute transport, cell migration, cell proliferation and differentiation.

In one embodiment, the ion channel or receptor type to be modulated is an N-type calcium channel or an L-type calcium channel. The N-type calcium channel may be an alpha(2)delta calcium channel subunit. In another embodiment, the L-type calcium channel may be Caᵥ0.2. Compounds that modulate N-type calcium channels may be useful in the treatment or amelioration of pain indications. On the other hand, compounds that modulate L-type calcium channels may be useful in the treatment or amelioration of a variety of cardiac diseases.

Hyperpolarisation-activated cation channels activate due to hyperpolarisation of the cell membrane. These channels are often sensitive to cyclic nucleotides such as cAMP and cGMP and may be permeable to ions such as potassium ions and sodium ions. These channels assist in the propagation of an action potential. In one embodiment, the hyperpolarisation-activated cation channel is hyperpolarisation-activated cyclic nucleotide-gated potassium channel 1 (HCN1), hyperpolarisation-activated cyclic nucleotide-gated potassium channel 2 (HCN2), hyperpolarisation-activated cyclic nucleotide-gated potassium channel 3 (HCN3), or hyperpolarisation-activated cyclic nucleotide-gated potassium channel 4 (HCN4).

Sodium channels are integral membrane proteins, and in cells such as neurons, sodium channels play a key role in the production of action potentials. Consequently, compounds affecting sodium channel function will generally have a more direct and significantly greater impact on the action potential of the biological cell than those compounds affecting calcium and chloride channel function. In one embodiment, the sodium channel is a Naᵥ1.1 channel (voltage gated sodium channel, type I, alpha subunit; gene: SCN1A), a Naᵥ1.2 channel (voltage gated sodium channel, type II, alpha subunit; gene: SCN2A), a Naᵥ1.3 channel (voltage gated sodium channel, type III, alpha subunit; gene: SCN3A), a Naᵥ1.4 channel (voltage gated sodium channel, type IV, alpha subunit; gene: SCN4A), a Naᵥ1.5 channel (voltage gated sodium channel, type V, alpha subunit; gene: SCN5A), a Naᵥ1.6 channel (voltage gated sodium channel, type VIII, alpha subunit; gene: SCN8A), a Naᵥ1.7 channel (voltage gated sodium channel, type IX, alpha subunit; gene: SCN9A); a Naᵥ1.8 channel (voltage gated sodium channel, type X, alpha subunit; gene: SCN10A); or a Naᵥ1.9 channel (voltage gated sodium channel, type XI, alpha subunit; gene: SCN11A). In another embodiment, the sodium channel is a Naᵥ1.5 channel. In a further embodiment, the sodium channel is a Naᵥ1.4 channel.

Potassium channels are known mainly for their role in repolarizing the cell membrane following action potentials. They effectively work to restore the cell membrane to its resting potential and to reprime sodium channels for subsequent action potential firing. For example, IKR and IKᵥLQT1 are known to be involved in repolarising the cell after an action potential. In one embodiment, the potassium channel is a neuronal potassium channel, a delayed rectifier potassium channel or an A-type potassium channel. In a further embodiment, the potassium channel is a Kᵥ4.2 channel (voltage gated potassium channel, Shal-related subfamily, member 2; gene: KCND2), a Kᵥ4.3 channel (voltage gated potassium channel, Shal-related subfamily, member 3; gene: KCND3), a IKᵥLQT1 channel (also known as Kᵥ7.1 channel; gene: KCNQ1), a hERG channel (also known as Kv11.1; gene: hERG (human Ether-á-go-go Related Gene or KCNH2)), a Kᵢᵣ2.1 channel (an inward rectifier potassium channel; gene: KCNJ2), a Kᵢᵣ2.2 channel (an inward rectifier potassium channel; gene: KCNJ12), a Kᵢᵣ2.3 channel (an inward rectifier potassium channel; gene: KCNJ4), a minK channel (voltage gated potassium channel, ISK-related family, member 1; gene: KCNE1), a MiRP1 channel (voltage gated potassium channel, ISK-related family, member 2; gene: KCNE2), a MiRP2 channel (voltage gated potassium channel, ISK-related family, member 3; gene: KCNE3) or a MiRP3 channel (voltage gated potassium channel, ISK-related family, member 4; gene: KCNE4). In another embodiment, the potassium channel is a IKᵥLQT1 channel.

In one embodiment, the potassium channel is a leak channel. Leak channels are also known as tandem-pore-domain potassium channels, and are known to comprise approximately 15 members. These channels are regulated by a number of factors including oxygen tension, pH, mechanical stretch and G-proteins.

In the case of an action potential, as the membrane potential increases, both the sodium and potassium channels begin to open. This process increases the passage of sodium ions into the cell and the balancing passage of potassium ions out of the cell. For small changes in membrane potential, the flow of potassium ions will overcome the flow of sodium ions and the membrane potential will return to its resting potential. However, if the voltage increases past a critical threshold, the flow of sodium ions suddenly increases and will temporarily exceed the flow of potassium ions, resulting in a condition whereby the positive feedback from the flow of sodium ions activates even more sodium channels. Thus, the cell produces an action potential.

Therefore, in most cases the sodium and potassium channels are directly responsible for regulating the flow of ions across the cell membrane, which causes the firing of an action potential and the restoration of the cell membrane after the event.

In the development of pharmaceuticals, the testing of the interactions between compounds and, for instance, the firing of neurons, is a particularly important step in obtaining approval for new pharmaceuticals. Adverse effects are a barrier in the development of new pharmaceuticals, particularly those that affect the functioning of the heart and brain.

Ion channels or receptors that should not be affected by potential pharmaceuticals may include, for example, the hERG channel, the IKR channel, the IKᵥLQT1 channel, Na_{V}1.5 channel and the MiRP1 channel. In one embodiment, the ion channel or receptor type that is functional is a hERG channel, a IKR channel, a IKᵥLQT1 channel or a MiRP1 channel.

In a further embodiment, the ion channel is the hERG channel, which is an ion channel of particular interest in testing pharmaceuticals for adverse effects. The hERG channel (which is encoded by human Ether-á-go-go Related Gene) is a pore-forming (a pore is the portion of the ion channel that opens to allow movement of ions) voltage-gated potassium channel, which is expressed in the heart and nervous tissue. In certain circumstances, the hERG channel can make up the entirety of the channel that conducts the delayed rectifier current for repolarization of cell membranes around the heart; the current involved in the firing of ventricular myocytes (muscle fibre cells) including the purkinje fibres.

Very small changes in hERG channel function can reduce the ability of the heart to operate properly. Consequently, it is vital to the approval of compounds for therapeutic use that they be shown not to adversely affect the hERG channel. Some compounds, for example, have been found to have the effect of mirroring a condition representative of illness such as is seen in the genetic mutation of the hERG channel, leading to Long QT (where Q and T are regular points on an electrocardiogram (ECG)—see FIG. 6a) syndrome—where the heart develops an arrhythmia which can lead to sudden death and cardiac arrest, seen as an elongation of the QT interval on an ECG (see FIG. 6b). Accordingly, the possibility of undesirable interaction between hERG and a pharmaceutical compound of interest is necessary to avoid.

Present methods used for assaying compounds against their effect on the hERG channel can require the harvesting of one cell, containing the hERG channel, for each test desired to be performed. The cells are often taken from a dog such as a beagle. Accordingly, to perform such experiments the animals must be bred to ensure they are free from diseases that may alter results, the animal must be treated and killed, the cell extracted and the experiment set up. In addition, there can be considerable barriers to obtaining approval for such experiments and subsequently finding carriers of suitable cells. Methods according to preferred embodiments, as described herein, may remove the need for such experiments and also ameliorate some of the effects on results of variables that can be difficult to quantify, such as animal health and age.

It would be appreciated that when more functional ion channel or receptor types for providing a waveform are present in the cell (or part thereof), it is more difficult to determine which ion channel or receptor type is affected by the compound assayed. Accordingly, in one embodiment one ion channel or receptor type for providing a waveform is functional in the biological cell (or part thereof).

In another embodiment of the invention, the one or more ion channel or receptor types that are either not present or not functional in the biological cell (or part thereof) are one or more ion channels. In a further embodiment, the one or more ion channel or receptor types that are either not present or not functional in the biological cell (or part thereof) are one or more voltage-gated ion channels.

The ion channel that is either not present or not functional in the biological cell (or part thereof) may be selected from the group consisting of a sodium channel, a potassium channel, a calcium channel, a chloride channel or a hyperpolarisation-activated cation channel. In one embodiment, the ion channel not present or not functional is a sodium channel. In another embodiment, the ion channel not present or not functional is a potassium channel. In a further embodiment, the ion channel not present or not functional is a calcium channel. Any, or combinations of, the channels to be modulated as discussed above, may also not be present or not functional in the biological cell (or part thereof).

It is to be understood that assays performed in accordance with the invention includes, for example, an experiment at a single concentration to determine whether a compound is active, in addition to multiple experiments at a variety of concentrations so as to obtain a dose response curve.

Using these assays, compounds that modulate ion channel or receptor types may be identified, and/or the activity of these compounds determined. The compounds to be tested could be produced synthetically, or through biological processes. Mixtures of compounds may also be tested, which may, for example, include testing of biological samples or extracts thereof.

While the compounds assayed may be new pharmaceuticals, they may also be used in the development of new pharmaceuticals or new lead compounds. For example, in one embodiment a range of similar compounds could be assayed according to the method of the invention to develop a pharmacophore for the receptor or ion channel assayed, assisting in the development of new pharmaceuticals.

Using the method according to the present invention, new pharmaceuticals for a wide variety of diseases or conditions may be identified. For example, such diseases or conditions may include, but are not limited to, arrhythmia, short QT syndrome, long QT syndrome, pain, neuropathic pain, fibromyalgia, epilepsy, cognition and memory disorders, movement disorders, affective disorders, mood disorders, skeletal muscle diseases, smooth muscle diseases, blood pressure and tremors.

The above method allows rapid development of virtual conductance models and the ability to incorporate graphical tools in the control of experiments and the analysis of data. As this analysis includes the fitting of real conductance models that include the effects of compounds on waveforms, it may be used to select from candidate compounds those compounds suitable for further experimentation or use. This selectivity also includes the forecasting of the effects of the compounds on other parts of the anatomy (i.e. a compound treating arrhythmia may also be suitable for the treatment of problems in other parts of the body, and such advantageous use, or disadvantageous use in the case of adverse effects, can potentially be forecast) and the guiding of medicinal chemists in their experimentations and compound selection.

### Examples

Human embryonic kidney (HEK) cells which stably express skeletal muscle Naᵥ1.4 sodium channels were obtained as a gift from Professor Holger Lerche at the University of Ulm, Germany. The creation and characterization of these cells is described in Mitrović et al., (1994) J Physiol., 478(Pt 3), 395-402. For maintenance, cells were cultured in Dulbecco's Modified Eagle Medium with 10% Fetal Bovine Serum in 144 cm² flask and incubated at 37° C. in 5% CO₂.

Twenty-four hours prior to experimentation, cells were dissociated using Versene (EDTA) and plated at 10-12% confluency onto coverslips. The following day the coverslips were placed into the recording chamber and held at 22-25° C. for the duration of the experiments.

Borosilicate glass pipettes (WPI) were used for the whole cell assay. These pipettes were filled with an intracellular solution containing (mM): 10 NaF, 110 CsF, 20 TEA.Cl, 2 ethylene glycol tetraacetic acid and 10 HEPES, with pH adjusted to 7.4 using CsOH and osmolarity adjusted to 310 mosmol/L with sucrose. The pipettes, when filled with this solution, had a resistance of 2-6 MOhms.

The bath solution contained in (mM): 141 NaCl, 4 KCl, 1.0 MgCl₂, 1.8 CaCl₂, 10 HEPES buffer, and 4 tetraethylammonium (TEA).Cl, with pH adjusted to 7.4 with NaOH and osmolarity adjusted to 310 mosmol/L with sucrose. Bath temperature was controlled using a Warner Instruments (Hamden, Conn.) controller (TC-344B) with inline solution and bath heating.

Electrophysiological recordings were made 10 minutes after establishing whole cell recording. Recordings were made on an EPC-9 patch clamp amplifier (Heka Instruments, Lambrecht, Germany) filtered at 14.4 kHz with >80% series resistance compensation and sampled at 50 kHz. The current monitor output from the EPC-9 was fed into the analogue input channel of a data acquisition card.

The dynamic clamp system was implemented in Simulink with Realtime workshop and the xPC target toolkit (see FIG. 7; All products from Mathworks). The model was compiled and downloaded to the target on a standard PC with a National Instruments PCI-6052E data acquisition board. The model runs in polling mode using the ode5 fixed time step solver with a step size of 50 μS.

The dynamic clamp system was configured to account for leak conductance, and to also simulate the function of potassium channels, which were not present in the HEK cell.

Leak current is given by:

I_{Leak}=g_{Leak}×(V−V_{Leak})

V_{Leak}=−85 mV

The fast delayed rectifier potassium current is given by Cannon et al. (1993) Biophys J., 65(1), 270-88:

I_{Kr}=g_{Kr}×n⁴×(V−Vₖ)

$$\frac{d n}{d t} = α ( V ) \times ( 1 - n ) - β ( V ) \times n$$

$$α ( V ) = \frac{{\overline{α}}_{n} \times ( V - {V}_{n} )}{1 - {e}^{- ( V - {V}_{n} ) / {K}_{α n}}}$$

β(V)= β̅ₙ×e^{-(V-V}^{ₙ}^{)/K}^{_{βn}}

### Vₖ=−93.1320 mV

α̅ₙ=0.0131/ms/mV
K_{αn}=7 mV
K_{βn}=40 mV
β̅ₙ=0.067/ms

### Vₙ=−40 mV

Following attainment of a whole cell clamp in the HEK cell, a period of 10 minutes was allowed for diffusion of the pipette solution into the intracellular volume of the cell. During this period cells were held at −85 mV.

Control was transferred to the Simulink system and a range of current injections were trialled to achieve a steady state action potential firing of 50-100 Hz (FIG. 8). This firing was stable and continued as long as a stimulating current injection was maintained.

Following a period of stable recording of action potential firing, 50 μM carbamazepine (CBZ, Sigma-Aldrich C8981, a sodium channel blocker) was added to the bath solution and perfused onto the cells. This decreased the action potential firing rate. FIG. 9 is an output of the simulator, showing the response of the system to a step of stimulating current. In the continued presence of 50 μM CBZ a stimulating current step elicited only 2-3 action potentials and no further firing would occur.

This shows that a dynamic clamp in electrical contact with a cell expressing sodium channels may be used to assist in producing and monitoring consecutive waveforms (action potentials) at that cell. Furthermore, it is illustrated that by modifying this system by modulating these sodium channels with a compound, the resultant waveform generated is affected.

The described constructions have been advanced merely by way of example and many modifications and variations may be made without departing from the spirit and scope of the invention, which includes every novel feature and combination of features herein disclosed.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge.

1. A method of assaying a compound for its ability to modulate an ion channel or receptor type, the method comprising:
a) providing a dynamic clamp in electrical contact with a biological cell (or part thereof) in which one or more ion channel or receptor types for providing a waveform are functional and in which one or more ion channel or receptor types for providing a waveform are either not present or not functional;
b) causing the dynamic clamp to apply a signal simulating the function of at least one of the one or more ion channel or receptor types that are either not present or not functional in the biological cell (or part thereof) based on modulation of the ion channel or receptor types that are functional in the biological cell (or part thereof) to thereby provide the waveform at the biological cell (or part thereof);
c) exposing at least one of the one or more functional ion channel or receptor types to a compound; and
d) detecting modulation of the waveform at the biological cell (or part thereof), wherein modulation of the waveform is indicative of a compound that modulates the at least one functional ion channel or receptor types.

2. The method according to claim 1, wherein the waveform is an action potential.

3. The method according to claim 1, wherein the dynamic clamp applies a voltage signal to the biological cell (or part thereof), and wherein modulation of the waveform at the biological cell (or part thereof) is detected by measuring a current signal at the biological cell (or part thereof).

4. The method according to claim 1, wherein the dynamic clamp applies a current n of the waveform at the biological cell (or part thereof) is detected by measuring a voltage signal at the biological cell (or part thereof).

5. The method according to claim 1, wherein the one or more ion channel or receptor types that are functional in the biological cell (or part thereof) are one or more ion channels.

6. The method according to claim 5, wherein the ion channel that is functional is selected from the group consisting of a sodium channel, a potassium channel, a calcium channel, a chloride channel or a hyperpolarisation-activated cation channel.

7. The method according to claim 1, wherein the ion channel or receptor type that is functional is a hERG channel, a IKR channel, a IKᵥLQT1 channel or a MiRP1 channel.

8. The method according to claim 1, wherein one ion channel or receptor type for providing a waveform is functional in the biological cell (or part thereof).

9. The method according to claim 1, wherein the one or more ion channel or receptor types that are either not present or not functional in the biological cell (or part thereof) are one or more ion channels.

10. The method according to claim 9, wherein the ion channel that is not present or not functional is selected from the group consisting of a sodium channel, a potassium channel, a calcium channel or a chloride channel.

11. The method according to claim 1, wherein the one or more ion channel or receptor types for providing a waveform are functional as they are expressed in the biological cell (or part thereof), and wherein the one or more ion channel or receptor types for providing a waveform are either not present or not functional as they are not expressed in the biological cell (or part thereof).

12. The method according to claim 1, wherein the biological cell (or part thereof) is selected from the group consisting of: a human embryonic kidney (HEK) cell, a COS cell, an LTK cell, a Chinese hamster lung cell, a Chinese hamster ovary (CHO) cell, or a Xenopus oocyte.

13. The method according to claim 1, wherein the biological cell (or part thereof) is a HEK cell.

14. (canceled)

15. An apparatus for assaying a compound's ability to modulate an ion channel or receptor type in a biological cell (or part thereof), the apparatus including:
a) One or more electrodes adapted to be provided in electrical contact with the biological cell (or part thereof), wherein the one or more electrodes are configured:
i. to detect modulation of one or more functional ion channels or receptor types for providing a waveform at the biological cell (or part thereof) and to provide a first signal based on the detected modulation; and
ii. to apply a second signal to the biological cell (or part thereof);
b) A simulator to simulate the function of at least one or more ion channel or receptor types for providing a waveform that are either not present or not functional in the biological cell (or part thereof);
i. wherein the simulator is configured to receive the first signal from the one or more electrodes and to provide the second signal to the one or more electrodes;
ii. wherein the second signal simulates the function of at least one of the one or more ion channel or receptor types that are either not present or not functional based on the first signal, to thereby provide the waveform at the biological cell (or part thereof).

16. The apparatus according to claim 15, wherein the simulator comprises an output to display at least one of a waveform or other data to allow a compound's ability to modulate an ion channel or receptor type to be determined.

17. The apparatus according to claim 15, wherein the simulator comprises one or more amplifiers.

18. The apparatus according to claim 17, wherein the simulator comprises a suitably programmed computing system

19. The apparatus according to claim 18, wherein the computing system operates to control the amplifier to provide the second signal to the one or more electrodes, and wherein the computing system operates to receive the first signal from the one or more electrodes.

20. The apparatus according to claim 19, wherein the computing system operates to analyse the first signal and control the amplifier in accordance with analysis of the first signal.

21. An apparatus for assaying a compound for its ability to modulate an ion channel or receptor type, the apparatus including:
a) One or more electrodes to measure an electrophysiological parameter at a biological cell (or part thereof) and to control a current or voltage applied to the biological cell (or part thereof), wherein the one or more electrodes are adapted for electrical connection with the biological cell (or part thereof);
b) One or more amplifiers to assist in measuring the electrophysiological parameter at the biological cell (or part thereof) and to assist in controlling the current or voltage applied to the biological cell (or part thereof), wherein the one or more amplifiers are electrically connected to the one or more electrodes; and
c) Software to simulate the function of one or more ion channel or receptor types in a biological cell (or part thereof), which function is simulated by receiving the measurement of the electrophysiological parameter at the biological cell (or part thereof) from the one or more amplifiers, determining the current or voltage to be applied to the biological cell (or part thereof) based on said measurement, and transmitting an electrical signal to the one or more amplifiers to control the current or voltage applied to the biological cell (or part thereof).

22. A process, including:
receiving data detected from the modulation of at least one ion channel or receptor type at a biological cell (or part thereof);
processing the data to determine a signal to be applied to the biological cell (or part thereof), wherein the signal represents one or more ion channel or receptor types that are either not functional or not present in the biological cell (or part thereof); and
applying the signal to the biological cell (or part thereof).

23. A computer-readable storage medium having stored thereon programming instructions for performing a process including:
receiving data detected from the modulation of at least one ion channel or receptor type at a biological cell (or part thereof);
processing the data to determine a signal to be applied to the biological cell (or part thereof), wherein the signal represents one or more ion channel or receptor types that are either not functional or not present in the biological cell (or part thereof); and
applying the signal to the biological cell (or part thereof).

24. A system configured to perform a process including:
receiving data detected from the modulation of at least one ion channel or receptor type at a biological cell (or part thereof);
processing the data to determine a signal to be applied to the biological cell (or part thereof), wherein the signal represents one or more ion channel or receptor types that are either not functional or not present in the biological cell (or part thereof); and
applying the signal to the biological cell (or part thereof).
